(19) United States
(12) Patent Application Publication
(10) Pub. No.: US 2023/0355260 A1
(43) Pub. Date: Nov. 9, 2023

(54) ARRANGEMENT OF END EFFECTOR ELEMENTS

(71) Applicant: CMR Surgical Limited, Cambridge (GB)

(72) Inventors: Ben Robert Chaplin, Cambridge (GB); David William Haydn Webster-Smith, Cambridge (GB)

(73) Assignee: CMR Surgical Limited, Cambridge (GB)

(21) Appl. No.: 18/044,913

(22) PCT Filed: Sep. 21, 2021
(86) PCT No.: PCT/GB2021/052448

§ 371 Date: Mar. 10, 2023

(30) Foreign Application Priority Data
Sep. 23, 2020 (GB) 2015015.7

(51) Int. Cl. A61B 17/28; B25J 15/00; A61B 34/30

(52) U.S. Cl. CPC A61B 17/2812; B25J 15/00; A61B 34/30

(57) ABSTRACT

A robotic surgical instrument comprising: a shaft; an end effector comprising a first end effector element with a first surface and a second end effector element with a second surface configured to interface with the first surface; and an articulation connecting the end effector to the shaft, the articulation permitting the first end effector element to rotate about a first axis and the second end effector element to rotate about a second axis, the first and second axes being transverse to the longitudinal axis of the shaft; wherein, when the end effector is aligned with the shaft and the first and second surfaces are interfaced, the orientation of the first surface, relative to the first axis, is greater than zero degrees.

### FIELD OF THE INVENTION

This invention relates to the angling of end effector elements in a robotic surgical instrument.

### BACKGROUND OF THE INVENTION

Surgical robots are commonly being used to perform surgical procedures, due to the improvements in precision and sterility that they offer when compared to manual open or laparoscopy operations. A typical surgical robot comprises a base unit, a robot arm, and a surgical instrument. The robot arm is connected at its proximal end to the base unit, and at its distal end to the surgical instrument. The surgical instrument, at its distal end, comprises an end effector for penetrating the body of a patient at a port to reach a surgical site where it engages in a medical procedure.

Advancements are continuously being made to improve on existing configurations of surgical instruments to be used in combination with operational surgical robots. Important factors to consider when devising these advancements include ensuring that motion demanded by a surgeon at a command interface is precisely transferred to motion of the end effector at the surgical site, and also that the drive efficiency of the instrument is maximised. The drive efficiency of a surgical instrument may be defined as the percentage of force applied to the instrument by a drive source (such as a motor) that is output by the end effector. The drive efficiency of the instrument can be used as an indicator of its general performance.

It is desirable to devise a surgical instrument which provides maximum precision and drive efficiency improvements.

### SUMMARY OF THE INVENTION

According to a first aspect, there is provided a robotic surgical instrument comprising: a shaft; an end effector comprising a first end effector element with a first surface and a second end effector element with a second surface configured to interface with the first surface; and an articulation connecting the end effector to the shaft, the articulation permitting the first end effector element to rotate about a first axis and the second end effector element to rotate about a second axis, the first and second axes being transverse to the longitudinal axis of the shaft; wherein, when the end effector is aligned with the shaft and the first and second surfaces are interfaced, the orientation of the first surface, relative to the first axis, is greater than zero degrees.

When the end effector is aligned with the shaft and the first and second surfaces are interfaced, the orientation of the second surface, relative to the first axis, may be greater than zero degrees.

The orientation of the first surface relative to the first axis may be between 20 and 35 degrees.

When the end effector is aligned with the shaft, the longitudinal axis of the end effector may be coincident with the longitudinal axis of the shaft.

The first end effector element may further comprise a third surface opposing the first surface, and the third surface may be parallel to the first surface.

The second end effector element may further comprise a fourth surface opposing the second surface, and the fourth surface may be parallel to the second surface.

The first end effector element and the second end effector element may be independently rotatable about the first and second axes, respectively.

The articulation may comprise a first joint permitting the first end effector element to rotate about the first axis and a second joint permitting the second end effector element to rotate about the second axis.

The first end effector element may be drivable by a first pair of driving elements and the second end effector element may be drivable by a second pair of driving elements.

The first joint may comprise a first threaded axle, and the first effector element may comprise a threaded channel configured to interface with the first threaded axle.

The second joint may comprise a second threaded axle, and the second end effector element may comprise a threaded channel configured to interface with the second threaded axle.

The threaded axle may have a pitch diameter of between 0.3 mm and 2 mm.

The articulation may further comprise a third joint, the third joint permitting the end effector to rotate about a third axis transverse to the first and second axes.

A distal end of the shaft may be connected to the articulation and a proximal end of the shaft may be connected to a drive mechanism for driving the articulation.

The articulation may further comprise a supporting body that is connected to the first end effector element by the first joint, the second end effector element by the second joint and the shaft by the third joint.

The first surface may be contained within a first plane and the second surface may be contained within a second plane and, when the end effector is aligned with the shaft and the first and second surfaces are interfaced, the orientation of both the first plane and the second plane, relative to the first and second axes, may be greater than zero degrees.

The first axis may be the same as the second axis.

The first and second end effector elements may be opposing first and second jaws of an end effector.

The first and second surfaces may be gripping surfaces.

The robotic surgical instrument may be configured to be connected to a surgical robot.

### BRIEF DESCRIPTION ON THE FIGURES

The present invention will now be described by way of example with reference to the accompanying drawings. In the drawings:

FIG. 1 illustrates a surgical robot;

FIGS. 2A and 2B illustrate first and second views of a first embodiment of a distal end of a surgical instrument;

FIG. 3 illustrates a third view of the first embodiment of the distal end of a surgical instrument;

FIG. 4 illustrates a fourth view of the first embodiment of the distal end of a surgical instrument;

FIG. 5 illustrates a first view of a second embodiment of a distal end of a surgical instrument;

FIG. 6 illustrates an alternative view of the second embodiment of the distal end of a surgical instrument;

FIGS. 7A, 7B and 7C illustrate the paths of motion for an improved embodiment of the distal end of the surgical instrument illustrated in FIGS. 4 and 5.

### DETAILED DESCRIPTION

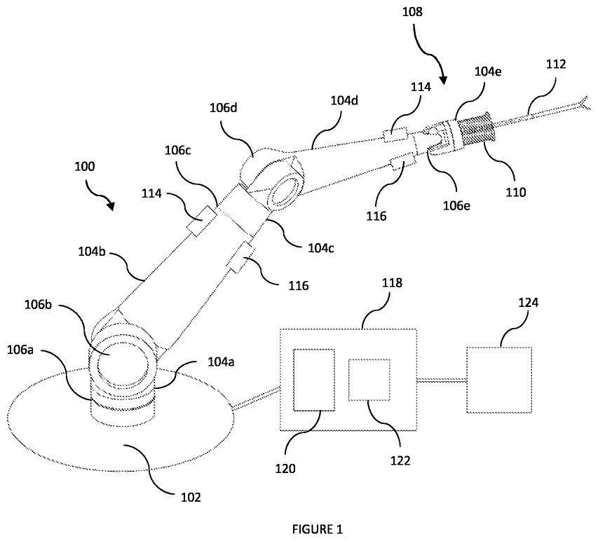

FIG. 1 illustrates a surgical robot having an arm 100 which extends from a base unit 102. The arm comprises a plurality of rigid limbs 104a-e which are coupled by a plurality of joints 106a-e. The joints 106a-e are configured to apply motion to the limbs. The limb that is closest to the base 102 is the most proximal limb 104a and is coupled to the base by a proximal joint 106a. The remaining limbs of the arm are each coupled in series by a joint of the plurality of joints 106b-e. A wrist 108 may comprise four individual revolute joints. The wrist 108 couples one limb (104d) to the most distal limb (104e) of the arm. The most distal limb 104e carries an attachment 110 for a surgical instrument 112. Each joint 106a-e of the arm 100 has one or more drive sources 114 which can be operated to cause rotational motion at the respective joint. Each drive source 114 is connected to its respective joint 106a-e by a drivetrain which transfers power from the drive source to the joint. In one example, the drive sources 114 are motors. The drive sources 114 may alternatively be hydraulic actuators, or any other suitable means. Each joint 106a-e further comprises one or more configuration and/or force sensors 116 which provides sensory information regarding the current configuration and/or force at that joint. In addition to configuration and/or force sensory data, the one or more sensors 116 may additionally provide information regarding sensed temperature, current or pressure (such as hydraulic pressure).

The arm terminates in an attachment for interfacing with the surgical instrument 112. The surgical instrument has a diameter less than 8 mm. The surgical instrument may have a 5 mm diameter. The surgical instrument may have a diameter which is less than 5 mm. The surgical instrument comprises an end effector for performing an operation. The end effector may take any suitable form. For example, the end effector may be smooth jaws, serrated jaws, a gripper, a pair of shears, a needle for suturing, a camera, a laser, a knife, a stapler, a cauteriser or a suctioner. The end effector may alternatively be an electrosurgical instrument such as a pair of monopolar scissors. The surgical instrument further comprises an instrument shaft and an articulation located between the instrument shaft and the end effector. The articulation comprises several joints which permit the end effector to move relative to the shaft of the instrument. The joints in the articulation are actuated by driving elements. These driving elements are secured at the other end of the instrument shaft to interface elements of the instrument interface. The driving elements are elongate elements that extend from the joints in the articulation through the shaft to the instrument interface. Each driving element can be flexed transverse to its longitudinal axis in the specified regions. For example, the driving elements may be cables.

The diameter of the surgical instrument may be the diameter of the profile of the articulation. The diameter of the profile of the articulation may match or be narrower than the diameter of the shaft. The attachment comprises a drive assembly for driving articulation of the instrument. Movable interface elements of the drive assembly interface mechanically engage corresponding movable interface elements of the instrument interface in order to transfer drive from the robot arm to the instrument. Thus, the robot arm transfers drive to the end effector as follows: movement of a drive assembly interface element moves an instrument interface element which moves a driving element which moves a joint of the articulation which moves the end effector.

Controllers for the drive sources 114 and sensors 116 are distributed within the robot arm 100. The controllers are connected via a communication bus to a control unit 118. The control unit 118 comprises a processor 120 and a memory 122. The memory 122 stores, in a non-transient way, software that is executable by the processor 120 to control the operation of the drive sources 114 to cause the arm 100 to operate. In particular, the software can control the processor 120 to cause the drive sources (for example via distributed controllers) to drive in dependence on inputs from the sensors 116 and from a surgeon command interface 124.

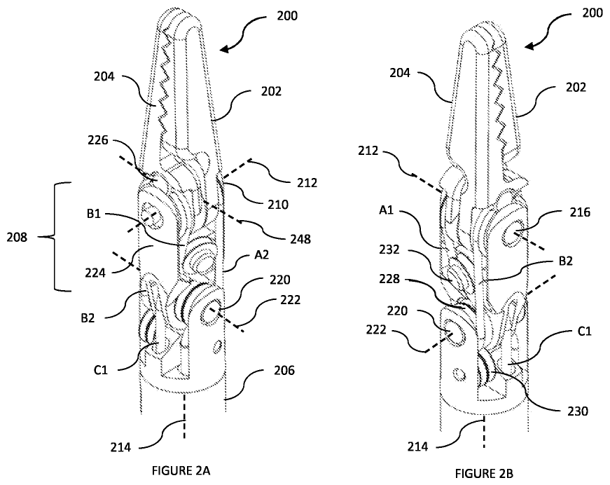

FIGS. 2A and 2B illustrate opposing views of the distal end of a surgical instrument for attachment to the arm of a surgical robot. The distal end of the surgical instrument comprises an end effector 200 with a pair of end effector elements 202, 204. The end effector 200 is connected to the distal end of the shaft 206 by an articulation 208. The shaft is connected at its proximal end to an interface for attaching to a robot arm. The drive mechanism may comprise a drive source as described with reference to FIG. 1 above. Articulation 208 comprises joints which permit movement of the end effector 200 relative to the shaft 206. A first joint 210 permits the first end effector element 202 to rotate about a first axis. The first axis is transverse to the longitudinal axis of the shaft 214. A second joint 216 permits the second end effector element 204 to rotate about a second axis. The second axis is also transverse to the longitudinal axis of the shaft 214. The second axis may be parallel to the first axis. In one example, the first and second axes are the same axis, as illustrated in FIGS. 2A to 7C by reference numerals 212 and 310. However, it would be understood by the skilled person that, in alternative examples, the second axis is not the same as the first axis. For example, the second axis may be parallel to but offset from the first axis. The offset may be in a direction defined by the longitudinal axis of the shaft, or alternatively in a direction that is transverse to the longitudinal axis of the shaft. The offset may be in an alternative direction that is not defined with respect to the longitudinal axis of the shaft.

The first end effector element 202 and the second end effector element 204 are independently rotatable about the first axis and the second axis respectively by the first and second joints. The end effector elements may be rotated in the same direction or different directions by the first and second joints. The first end effector element 202 may be rotated about the first axis, whilst the second end effector element 204 is not rotated about the second axis. The second end effector element 204 may be rotated about the second axis, whilst the first end effector element 202 is not rotated about the first axis. The shaft terminates at its distal end in the third joint 220. A third joint 220 permits the end effector 200 to rotate about a third axis 222. The third axis 222 is transverse to the first axis 212.

The articulation 208 comprises a supporting body 224. At a first end, the supporting body 224 is connected to the end effector 200 by the first joint 210 and the second joint 216. At a second end opposing the first end, the supporting body 224 is connected to the shaft 206 by the third joint 220. The first joint 210 and second joint 216 permit the end effector elements 202, 204 to rotate relative to the supporting body 224 about the first and second axes 212. The third joint 220 permits the supporting body 224 to rotate relative to the shaft 206 about the third axis 222.

In FIGS. 2A and 2B the surgical instrument is in a straight configuration. In this configuration, the end effector 200 is aligned with the shaft 206. That is, the longitudinal axis of the articulation and the longitudinal axis of the end effector are coincident with longitudinal axis 214 of the shaft. The first and second axes are both transverse to the longitudinal axis 214 of the shaft. Articulation of the first, second and third joints enables the end effector to take a range of attitudes relative to the shaft.

Each joint of the end effector is driven by a pair of driving elements. So, each joint is independently driven. The first joint 210 is driven by a first pair of driving elements A1, A2. The second joint 216 is driven by a second pair of driving elements B1, B2. The third joint 212 is driven by a third pair of driving elements C1, C2 (not visible). At one point, driving elements of a pair of driving elements are secured to their corresponding joint. For example, the second pair of driving elements B1, B2 comprises a ball feature 226 which is secured to the second joint 216. This ensures that when the pair of driving elements is driven, the drive is transferred to motion of the joint about its axis.

The surgical instrument of FIGS. 2A and 2B further comprises a pulley arrangement around which the first and second pairs of driving elements are constrained to move. The pulley arrangement comprises a first set of pulleys 228 rotatable about the third axis 222. That is, the first set of pulleys 228 rotate about the same axis as the third joint 220. The pulley arrangement further comprises at least a second set of pulleys 230 and a pair of redirecting pulleys 232. The pulley arrangement, and the routing of driving elements around this arrangement, may correspond to the arrangement described in PCT application no.: WO 2017/098279 A1.

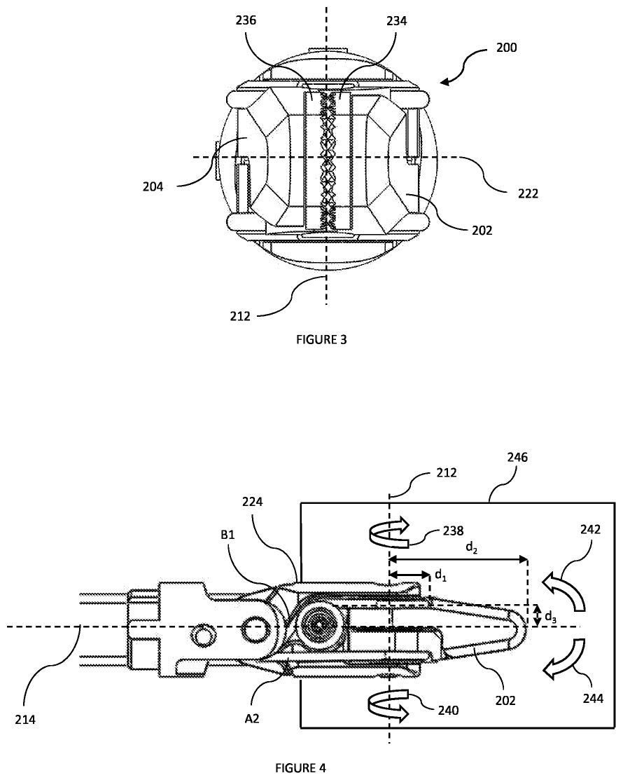

The end effector elements 202, 204 are illustrated in FIGS. 2A and 2B as a pair of opposing serrated jaws. However, the end effector elements may take any alternative form. Alternative views of the surgical instrument of FIGS. 2A and 2B are illustrated in FIGS. 3 and 4. So, the joints and pulley arrangement of the instrument illustrated in FIGS. 3 and 4 correspond to those described above with reference to FIGS. 2A and 2B. In FIGS. 3 and 4 the surgical instrument is configured such that the end effector is aligned with the shaft. That is, the longitudinal axis is of the end effector is coincident with the longitudinal axis of the shaft.

FIG. 3 illustrates the end effector 200 as viewed from its distal end. The distal end of the end effector is the end furthest from the shaft 206. FIG. 4 illustrates a lateral view of the end effector 200. The first end effector element 202 of the end effector 200 comprises a first surface 234. The second end effector element 204 comprises a second surface 236. The first and second surfaces of the first and second end effector elements are configured to interface. That is, the first and second surfaces are configured to contact each other when the first and second end effector elements are in a closed configuration. The whole of the first surface is configured to contact the whole of the second surface in the closed configuration. The first and second surfaces may otherwise be referred to as the inner surfaces of the first and second end effector elements, as they are located inside the end effector when they are interfaced. The first and second surfaces may be gripping surfaces. That is, the first and second surfaces may be configured to interface such that they can grip an object located between them. The object may be a needle. The first and second axes 212 about which the first and second joints rotate lie on a plane at which the first surface 234 and the second surface 236 meet. In the configuration illustrated in FIGS. 3 and 4, where the end effector 200 is aligned with the shaft 206, the first and second axes 212 are transverse to the longitudinal axis of the shaft. Together, the longitudinal axis 214 and the first and second axes 212 define a contact plane between the first surface 234 and the second surface 236.

The first surface 234 of the end effector element is located in a first plane 246. The second surface 236 of the end effector element is located in a second plane (not illustrated). When the end effector is aligned with the shaft and the first and second surfaces are interfaced as illustrated in FIG. 3, the first and second axes 212 lie on the first plane containing the first surface. That is, the orientation of the first plane relative to the first and second axes 212 is zero degrees. Correspondingly, when the surfaces are interfaced, the first and second axes 212 lie on the second plane containing the second surface. That is, the orientation of the second plane relative to the first and second axes 212 is zero degrees. In other words, the first and second planes extend in a first direction parallel to the first and second axes 212. The first and second planes extend in a second direction parallel to the longitudinal axis of the shaft.

The first and second planes are not illustrated in FIG. 3 as they extend parallel to the first and second axes 212. So, the first direction of the first and the second planes, in FIG. 3, correspond to the direction of the first and second axes 212. The first plane and the second plane may be the same plane.

Where the first plane is described as containing the first surface, this plane contains an average line representing the orientation of the first surface as it extends in a first direction. Where the second plane is described as containing the second surface, this plane contains an average line representing the orientation of the second surface as it extends in the first direction. The first and second surfaces may be planar surfaces. In this example, the average line is the same as the orientation of the first surface across its length in the first direction. That is, the orientation of the first surface does not vary across its length in the first direction and so the first surface is entirely contained within the first plane. For corresponding reasons, the second surface is entirely contained within the second plane. The first and second surfaces may alternatively be non-planar surfaces. In this example, the average line for the first surface represents the average orientation of the first surface as it extends in the first direction. The average line for the second surface represents the average orientation of the second surface as it extends in the first direction. The first direction, in these examples, is transverse to the longitudinal axis of the shaft when the end effector 200 is aligned with the shaft 206.

The end effector is actuated by applying tension to one or more of the driving elements driving the joints in the articulation 208. The tension is applied by one or more corresponding drive sources located in the robot arm and configured to drive each of the joints in the articulation. A first driving element of a pair of driving elements is pulled to rotate a corresponding end effector element around its corresponding axis in a first direction. A second driving element of a pair of driving elements is pulled to rotate the corresponding end effector element around its corresponding axis in an opposite direction. For example, pulling driving element A1 will cause the rotation of the first end effector element 202 about the first axis 212 in a first direction 238. Pulling driving element A2 will cause the rotation of the first end effector element 202 about the first axis 212 in a second direction 240 opposing the first direction. The second joint is similarly actuated.

The application of tension to either of driving elements A1, A2 results in a first moment that causes first end effector element 202 to rotate about the first axis. The application of tension to either of driving elements B1, B2 results in a second moment that causes the second end effector element 204 to rotate about the second axis. The first direction in which the end effector elements are configured to rotate about the first and second axes 212 is illustrated by reference 238. The second direction 240 opposes this direction. The first and second moments may be defined as the “intended” moments for the end effector. That is, rotation of the end effector elements 202, 204 about the first and second axes 212 results in opening and closing of the end effector. When the end effector is in a closed configuration, the first surface 234 and the second surface 236 are interfacing. In other words, the first and second surfaces are contacting, or meeting, each other.

The first moment is quantified as a first distance d₁ multiplied by the tensile force T₁ applied by tension in either of driving elements A1, A2. The first distance d₁ is defined as the distance between the attachment point of the end effector element to its corresponding driving elements, and the rotational axis of the end effector. The attachment point of the end effector to its driving elements corresponds to the location of ball feature 226 around which the second pair of driving elements B1, B2 is secured. The distance d₁ therefore corresponds approximately to the radius of the pulley around which the first and second pair of driving elements rotate. The ball feature 226 for each pair of driving elements is able to rotate with its respective end effector element as that end effector rotates about the first, second and third axes. The first distance d₁ is variable in accordance this rotation. The second moment is quantified as the first distance d₁ multiplied by the corresponding tensile force T₂ applied by tension in either of driving elements B1, B2.

In addition to the first and second moments, the first and second end effector elements 202, 204 experience additional moments about axes transverse to the first and second axes 212. More specifically, the first and second end effector elements 202, 204 experience third and fourth moments respectively about a fourth axis 248, as illustrated in FIG. 2A. The fourth axis 248 is parallel the third axis 222, but intersects the first and second axes 212. The third and fourth moments may be referred to as “unintended” moments of the end effector elements. These “unintended” moments result in the tilting or rocking of the end effector elements around the first and second joints 210, 216.

FIG. 4 illustrates a third distance d₃ that represents the length of the first joint. The third distance d₃ additionally represents the length of the second joint. The lengths of the first and second joints extend from the longitudinal axis of the shaft 214 to an interior surface of the supporting body 224, and parallel to the first and second axes 212. The third moment acts on the first end effector element 202, and is quantified as the third distance d₃, multiplied by the tensile force T₁ applied by either of driving elements A1, A2. The fourth moment acts on the second end effector element 204, is quantified as the third distance d₃, multiplied by the corresponding tensile force T₂ applied by either of driving elements B1, B2. Depending on which end effector element is applying the tension, the third and fourth moments may either act in a first direction 238 or in a second direction 240 opposing the first direction.

In one example, the driving elements are tensioned to perform the closing motion on the end effector. As viewed from the first plane 246, when the end effector is in its closed configuration, the first and second end effector elements should preferably be aligned with the longitudinal axis of the shaft 214. This preferable configuration is illustrated in FIG. 4. During closing of the end effector, driving element A1 is tensioned to cause a first moment to rotate first end effector element 202 in the first direction 238 (towards the second end effector element 204). The tension in driving element A1 also results in a third moment rotating the first end effector element 202 in a third direction 242 away from the longitudinal axis of the shaft 214 (and about the fourth axis 248). Driving element B1 is tensioned to cause a second moment to rotate first end effector element 204 in the second direction 240 (towards the first end effector element 202). The tension in B1 also results in a fourth moment which acts in a fourth direction 244 away from the longitudinal axis of the shaft and opposing the third direction (also about the fourth axis 248). That is, the fourth direction is an opposite rotational direction to the third direction. So, as the end effector elements are pulled towards a closed configuration, the “unintended” moments are such that the end effector elements are pulled in opposing directions about the fourth axis 248, away from the longitudinal axis of the shaft. This results in a misalignment of the end effector elements as viewed from first plane 246, away from the preferable configuration illustrated in FIG. 4. The “unintended” moments result in a reduced efficiency of the end effector, as force applied by the drive source to the cables is lost to these moments. The efficiency of an end effector element, for the purposes of determining the effects of unintended moments, is quantified as the percentage of force output by that element relative to the tensile force that is generated by the driving elements that drive that end effector element. It is appreciated that the overall efficiency of the end effector is affected by many additional factors, such as friction around the pulleys and drive interfaces of the surgical instrument. Where the surfaces of the end effector elements are gripping surfaces, the force that is generated by each end effector element is a gripping force.

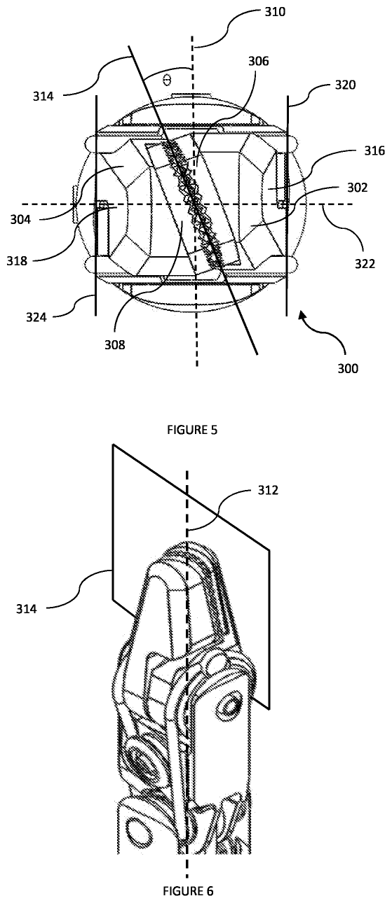

The abovementioned problem may be overcome by providing an end effector arrangement as illustrated in FIGS. 5 and 6. The joints and pulley arrangement of the surgical instrument comprising the end effector illustrated in FIGS. 4 and 5 correspond to those described with reference to FIGS. 2A, 2B, 3 and 4 above. In FIGS. 5 and 6 the surgical instrument is configured such that the end effector is aligned with the shaft. That is, the longitudinal axis is of the end effector is coincident with the longitudinal axis of the shaft.

As with the arrangement illustrated in FIGS. 2A, 2B, 3 and 4, the end effector comprises a first end effector element 302 and a second end effector element 304. The first end effector element comprises a first surface 306. The second end effector element comprises a second surface 308. The first and second surfaces of the first and second end effector elements are configured to interface, or in other words to contact when the first and second end effector elements are in a closed configuration. As mentioned above, the whole of the first surface is configured to contact the whole of the second surface in the closed configuration. The first and second surfaces may otherwise be referred to as the inner surfaces of the first and second end effector elements, as they are located inside the end effector when they are interfaced. The first and second surfaces may be gripping surfaces. That is, the first and second surfaces may be configured to interface such that they can grip an object located between the surfaces. The object may be a needle. The axis 310 separating the first surface and the second surface corresponds to the first and second axes 212 illustrated in FIGS. 2A, 2B, 3 and 4. That is, the first and second joints of the end effector rotate about the first and second axes 310. When the end effector is aligned with the shaft, the first and second axes 310 are transverse to the longitudinal axis 312 (i.e. 214) of the shaft. Together, the longitudinal axis 312 and the first and second axes 310 define a contact plane between the first surface 306 and the second surface 308.

The first surface 306 of the end effector element is located in a first plane 314. The second surface 308 is located in a second plane (not illustrated). In contrast to the example illustrated in FIG. 3, when the end effector is aligned with the shaft and the first and second surfaces are interfaced, the orientation of the first plane 314 containing the first surface 306, relative to the first and second axes 310, is greater than zero degrees. The orientation of the first plane 314 relative to the first and second axes 310 in a first direction is represented by θ in FIG. 5. When the first and second surfaces are interfaced, the orientation of the second plane (not illustrated) in a first direction relative to the first and second axes 310, is also greater than zero degrees. The first and second planes extend in a second direction parallel to the longitudinal axis of the shaft. The first plane and the second plane may be the same plane.

The configuration of the end effector elements as illustrated in FIGS. 5 and 6 is advantageous as it allows the otherwise “unintended” moments experienced by the end effectors to contribute towards the net force that they output. The force of the end effector elements can be calculated by dividing the moment that they experience by the effective length of the end effector element. For “intended” moments acting about the first and second axes 310, this effective length is the distance between the first and second axes and the distal end of the end effector (i.e. third distance d₂). For “unintended” moments acting about the fourth axis 248, the effective length varies as the end effector elements rotate about the first and second axes. The force that is output by the first end effector element 302 acts in a direction that is transverse to the first surface 306. The force that is output by the second end effector element 304 acts in a direction that is transverse to the second surface 308. By angling the end effector elements so that the first and second surfaces are not parallel to the first and second axes, the net force of the end effectors is a combination of a component of the force applied by the “intended” moment (which acts parallel to the first and second axes 212, 310) and a component of the force applied by the “unintended” moment (which acts about the fourth axis 248). The combination of the components of these two forces that act in the direction that is transverse to the surfaces 306, 308 of the end effector elements is greater than the force output by the end effector elements in the surgical instrument 200. In surgical instrument 200, it is only the component of the force acting parallel to the third axis 222, 322 that contributes to the net force output by the end effector elements.

By allowing the “unintended” moments to contribute to the net force output by the end effector elements, the value of this net force is increased for the end effector of surgical instrument 300 over that of surgical instrument 200. The efficiency of the end effector elements is also increased. The efficiency of an end effector element corresponds to the proportion of force output by that element relative to the tensile force that is generated by the driving elements that drive that end effector element.

The orientation of the first surface relative to the first and second axes, as viewed from the second plane may be between 20 and 35 degrees. It has been recognised by the inventors that this range of orientations allows for optimisation of the tilting force of the end effector elements that can be used to contribute to the net force of the end effector elements.

The first end effector element 302 further comprises a third surface, 316. The third surface is located on the exterior of the end effector, when the end effector elements are interfacing. That is, the third surface 316 opposes the first surface 306, which is located on the inside of the end effector when the end effector elements are interfacing. In FIG. 5, a third plane 320 containing the third surface 316 is parallel to the first and second axes 310. As the orientation of the first and second axes 310 relative to the first plane 314, is greater than zero degrees, or θ, the orientation of the third plane 320 relative to the first plane 314 is also greater than zero degrees, or θ. Correspondingly, the second end effector element 304 further comprises a fourth surface, 318. The fourth surface is located on the exterior of the end effector, when the end effector elements are interfacing. That is, the fourth surface 318 opposes the second surface 308, which is located on the interior of the end effector when the end effector elements are interfacing. In FIG. 5, a fourth plane 324 containing the fourth surface is parallel to the first and second axes 310. As the orientation of the first and second axes 310 relative to the first plane 314 is greater than zero degrees, the orientation of the fourth plane 324 relative to the first plane 314 is also greater than zero degrees (as indicated by θ in FIG. 5). The first plane is alternatively illustrated in FIG. 6.

A non-parallel orientation of the outer surfaces of the end effector elements relative to the inner surfaces of these elements may be misleading for a surgeon operating the surgical instrument. This is because the configuration of the outer surfaces may be used as a reference for the configuration of the inner surfaces. That is, the surgeon may use the outer surfaces of the end effector elements as a guide to indicate the orientation of the inner, gripping surfaces. So, in order to grip an object using the end effector illustrated in FIGS. 5 and 6, the surgeon would have to remember that the orientation of the inner surfaces of the end effector elements are not the same as the orientation of the outer surfaces.

In an alternative example of the arrangement illustrated in FIGS. 5 and 6, the third surface 316 of the first end effector element is parallel to its first surface 306. That is, the first plane containing the first surface is parallel to the second plane containing the third surface. Correspondingly, in this alternative example the fourth surface 318 of the second end effector element is parallel to its second surface 308. That is, the second plane containing the second surface is parallel to the fourth plane containing the fourth surface. This configuration is advantageous as it allows the surgeon to accurately visualise the alignment of the inner surfaces of the end effector elements from the configuration of their outer surfaces.

In addition to considering moments about the first and second axes 212, 310 and the fourth axis 248 of the shaft it may also be important to consider the vertical force component of the end effectors. The vertical force component acts in a direction that is perpendicular to the first and second axes. A further enhancement can be provided to maximise the force in this direction and to contribute to the net force of the end effector.

The first and second joints 210, 216 may be cylindrical pins with an extruded length and a consistent cross-sectional area along that length. The first and second joints 210, 216 may alternatively be a single cylindrical pin. However, to optimise the vertical force component contributing to the net force of the end effector, the cylindrical pins may be replaced with threaded axles. That is, the first joint 210 may comprise a first threaded axle, and the second joint 216 may comprise a second threaded axle. The diameter of the first threaded axle may be the same as the diameter of the second threaded axle. The first and second threaded axles may have a pitch diameter of between 0.3 mm and 2 mm. In one example, the first and second threaded axles may have a pitch diameter of 0.35 mm. This diameter of thread corresponds to an M1.6 thread. The movement of the end effectors along the first and second threaded axles is dependent on the distance between consecutive crests on these axles. The preferred ranges of pitch diameters selected herein are advantageous as they provide a suitable range of displacement for end effector elements along the first and second axes 212, given the range of angular motion required by the end effector. The first and second joints 210, 216 may alternatively be a single threaded axle.

The first and second end effector elements may also be threaded internally with a thread corresponding to the first and second threaded axles. That is, the first end effector element may comprise a first threaded channel configured to interface with the first threaded axle. The second end effector element may comprise a second threaded channel configured to interface with the second threaded axle. The diameter of the first threaded channel may be the same as the diameter of the second threaded channel. The internal threads of the first and second end effector elements enable them to be guided against the threaded axles of the first and second joints when they are rotated about the first and second axes 212, 310. The threaded channels may otherwise be defined as tapped holes. The diameters of the threaded channels are selected so that they correspond to the diameter of the first and second axles.

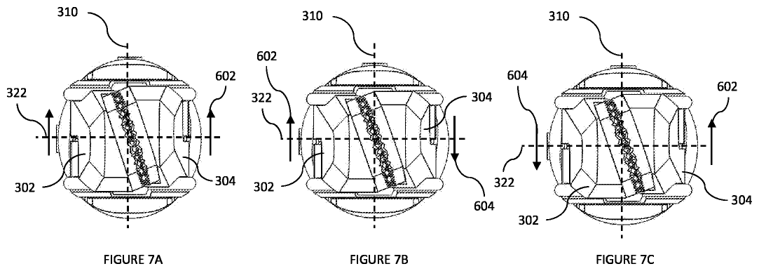

The threading of the first and second joints 210, 216 and corresponding channels in the end effector elements allows for vertical motion of the end effector elements to contribute to the net force exerted by those elements. Examples of how this motion is used are illustrated in FIGS. 7A, 7B and 7C.

FIG. 7A illustrates the motion of the end effector elements as the end effector is permitted to rotate about the first and second axes 310. In addition to permitting the first and second end effector elements to rotate, the first and second axes are also used to set the pitch angle of the end effector. The pitch angle is defined as the angle between the longitudinal axis of the shaft and the longitudinal axis of the end effector. That is, the pitch angle defines the orientation of the end effector as a whole relative to the shaft. When the pitch angle of the end effector is varied but the orientation of the first and second end effector elements with respect to each other remains the same, both end effectors will also move sideways along the first and second axes 310.

During the motion illustrated in FIG. 7A, both end effector elements move in the same direction along the first and second axes 310. So, as the end effector rotates in a first direction, if the first end effector element moves in a first direction 602 along the threaded axle of the first joint 210, then the second end effector element also moves in the first direction 602 along the threaded axle of the second joint 216. As the end effector rotates in a second direction opposing the first direction, the first end effector element and the second end effector element both move in a second direction opposing the first direction 602 along the joints 210, 216.

FIG. 7B illustrates the motion of the end effector elements as they are permitted to rotate about the first and second axes 310 by the first and second joints 210, 216 into a closed configuration. During this motion, the first end effector element 302 moves in a first direction 602 along the threaded axle of the first joint 210. The second end effector element 304 moves in a second direction 604 opposing the first direction along the threaded axle of the second joint 216. So, as the end effector elements are moved towards the closed configuration, they move towards each other along their respective threads. In this way, the movement of the end effector elements along their threads contributes to closing motion of the end effector.

FIG. 7C illustrates the motion of the end effector elements as they are permitted to rotate about the first and second axes 310 by the first and second joints 210, 216 towards an open configuration. During this motion, the first end effector element 302 moves in the second direction 604 along the threaded axle of the first joint 210. The second end effector element 304 moves in the first direction 602 opposing the first direction along the threaded axle of the second joint 216. So, as the end effector elements are moved towards the open configuration, they move away from each other along their respective threads. In this way, the movement of the end effector elements along their threads contributes to opening motion of the end effector.

The contribution of “unintended” moments to the overall force provided by the end effector 300 may be further increased by providing compliance, or relative motion, in the tilting motion between the first and second end effector elements. Compliance may be provided by widening the clearance between interfacing components of the end effector. That is, compliance may be provided between components of the end effector which increase the resistive forces and friction within the end effector when unintended moments are applied to those components. Compliance may be provided to widen the clearance between the first and second end effector elements. In one example, the clearance may be widened by the placement of cushions or spacers between the first and second end effector elements. The cushions and spacers are configured to deform in compression but maintain separation between the end effector elements when in a relaxed state. Compliance may also be provided, by means of cushions or spacers, between other components of the end effectors such as the supporting body or pulleys.

Whilst the specific examples in FIGS. 2A-7C illustrate the first axis and the second axis as being the same axis, it would be understood by the skilled person that, in alternative examples, the first and second axes may be different axes. For example, the second axis may be parallel to but offset from the first axis. The offset may be in a direction defined by the longitudinal axis of the shaft, or alternatively in a direction that is transverse to the longitudinal axis of the shaft. The offset may be in an alternative direction that is not defined with respect to the longitudinal axis of the shaft. In one example, where the first and second axes are different axes, the first and second surfaces of the end effector elements are orientated relative to only the first axis.

The examples in FIGS. 2A-7C illustrate end effectors with end effector elements that have substantially planar interfacing surfaces, such that these surfaces can be entirely contained within a first plane and a second plane respectively. It is mentioned above that, in alternative examples, the interfacing surfaces of the end effector elements may be non-planar. One example of a non-planar pair of end effector elements is a curved pair of monopolar scissors. The curved surfaces of the monopolar scissors do not extend linearly in a first direction that is transverse to the longitudinal axis of the shaft when the end effector is aligned with the shaft. For such end effector elements, it is the direction of the average line that is formed by the first and second surfaces as they extend in this first direction that is used to determine the orientation of the surfaces relative to the first and second axes. That is, the average line of the first and second surfaces as they extend in the first direction should correspond to the direction of the plane illustrated by reference 314 in FIG. 5.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A robotic surgical instrument comprising:
a shaft;
an end effector comprising a first end effector element with a first gripping surface and a second end effector element with a second gripping surface configured to interface with the first gripping surface, the first and second gripping surfaces being the respective inner surfaces of the first and second end effector elements; and
an articulation connecting the end effector to the shaft, the articulation permitting the first end effector element to rotate about a first axis and the second end effector element to rotate about a second axis, the first and second axes being transverse to the longitudinal axis of the shaft;
wherein, when the end effector is aligned with the shaft and the first and second gripping surfaces are interfaced, the orientation of the average line representing the orientation of the first gripping surface, relative to the first axis, is greater than zero degrees.

2. A robotic surgical instrument as claimed in claim 1 wherein,
when the end effector is aligned with the shaft and the first and second gripping surfaces are interfaced, the orientation of the average line representing the orientation of the second gripping surface, relative to the first axis, is greater than zero degrees.

3. A robotic surgical instrument as claimed in claim 1, wherein the orientation of the average line representing the orientation of the first gripping surface relative to the first axis is between 20 and 35 degrees.

4. A robotic surgical instrument as claimed in claim 1 wherein, when the end effector is aligned with the shaft, the longitudinal axis of the end effector is coincident with the longitudinal axis of the shaft.

5. A robotic surgical instrument as claimed in claim 1, wherein the first end effector element further comprises a third surface opposing the first gripping surface, and wherein the third surface is parallel to the first gripping surface.

6. A robotic surgical instrument as claimed in claim 1, wherein the second end effector element further comprises a fourth surface opposing the second gripping surface, and wherein the fourth surface is parallel to the second gripping surface.

7. A robotic surgical instrument as claimed in claim 1, wherein the first end effector element and the second end effector element are independently rotatable about the first and second axes, respectively.

8. A robotic surgical instrument as claimed in claim 1, wherein the articulation comprises a first joint permitting the first end effector element to rotate about the first axis and a second joint permitting the second end effector element to rotate about the second axis.

9. A robotic surgical instrument as claimed in claim 1, wherein the first end effector element is drivable by a first pair of driving elements and the second end effector element is drivable by a second pair of driving elements.

10. A robotic surgical instrument as claimed in claim 1, wherein the first joint comprises a first threaded axle, and the first effector element comprises a threaded channel configured to interface with the first threaded axle.

11. A robotic surgical instrument as claimed in claim 1, wherein the second joint comprises a second threaded axle, and the second end effector element comprises a threaded channel configured to interface with the second threaded axle.

12. A robotic surgical instrument as claimed in claim 10, wherein the threaded axle has a pitch diameter of between 0.3 mm and 2 mm.

13. A robotic surgical instrument as claimed in claim 8, wherein the articulation further comprises a third joint, the third joint permitting the end effector to rotate about a third axis transverse to the first and second axes.

14. A robotic surgical instrument as claimed in claim 1, wherein a distal end of the shaft is connected to the articulation and a proximal end of the shaft is connected to a drive mechanism for driving the articulation.

15. A robotic surgical instrument as claimed in claim 13, wherein the articulation further comprises a supporting body that is connected to the first end effector element by the first joint, the second end effector element by the second joint and the shaft by the third joint.

16. A robotic surgical instrument as claimed in claim 1, wherein the first gripping surface is contained within a first plane and the second gripping surface is contained within a second plane and, when the end effector is aligned with the shaft and the first and second gripping surfaces are interfaced, the orientation of the average line representing the orientation of both the first plane and the second plane, relative to the first and second axes, is greater than zero degrees.

17. A robotic surgical instrument as claimed in claim 1, wherein the first axis is the same as the second axis.

18. A robotic surgical instrument as claimed in claim 1, wherein the first and second end effector elements are opposing first and second jaws of an end effector.

19. A robotic surgical instrument as claimed in claim 1, wherein the robotic surgical instrument is configured to be connected to a surgical robot.
